(19) United States
(12) Patent Application Publication
(10) Pub. No.: US 2026/0014065 A1
(43) Pub. Date: Jan. 15, 2026

(54) PHYTOCHEMICAL INFUSED COSMETIC COMPOSITIONS AND THERAPEUTIC USES THEREOF

(71) Applicant: Dewey Beauty LLC, Pelham, AL (US)

(72) Inventor: Ciara Green, Pelham, AL (US)

(21) Appl. No.: 18/769,097

(22) Filed: Jul. 10, 2024

(51) Int. Cl. A61K 8/49; A61K 8/06; A61K 8/34; A61Q 1/02; A61Q 1/04; A61Q 1/06; A61Q 1/10

(52) U.S. Cl. CPC A61K 8/498; A61K 8/06; A61K 8/347; A61Q 1/02; A61Q 1/04; A61Q 1/06; A61Q 1/10; A61K 2800/43

(57) ABSTRACT

Color cosmetic infused with active phytochemical compounds, including cannabinoids endocannabinoids terpene or combinations of the same, and their methods of production and use.

### CROSS REFERENCES TO RELATED APPLICATION

This application claims the benefit of and priority to U.S. Provisional Application No. 63/525,803, filed Jul. 10, 2023. The entire specification and figures of the above-referenced application is hereby incorporated in its entirety by reference.

### TECHNICAL FIELD

The present invention relates generally to novel formulations and composition of matter comprising cosmetics, and preferably cannabinoid and/or terpenoid infused colored cosmetic compositions for aesthetic and therapeutic effect.

### BACKGROUND

Cannabinoids are a class of specialized compounds synthesized by Cannabis. They are formed by condensation of terpene and phenol precursors. They include these more abundant forms: Δ⁹-tetrahydrocannabinol (THC), cannabidiol (CBD), cannabichromene (CBC), and cannabigerol (CBG). Another cannabinoid, cannabinol (CBN), is formed from THC as a degradation product and can be detected in some plant strains. Typically, THC, CBD, CBC, and CBG occur together in different ratios in the various plant strains. These cannabinoids are generally lipophilic, nitrogen-free, mostly phenolic compounds and are derived biogenetically from a monoterpene and phenol, the acid cannabinoids from a monoterpene and phenol carboxylic acid and have a C21 base. Cannabinoids also find their corresponding carboxylic acids in plant products. In general, the carboxylic acids have the function of a biosynthetic precursor. For example, the tetrahydrocannabinols Δ⁹-and Δ⁸-THC arise in vivo from the THC carboxylic acids by decarboxylation and likewise, CBD from the associated cannabidiolic acid. Numerous health benefits have been note through the administration of one or more cannabinoids.

Terpenes, which may be used interchangeably with the term terpenoids, are a large and diverse class of naturally occurring organic chemicals. Terpenes are essential for plant metabolism, influencing general development, herbivory defense, pollination and stress response. These compounds have been extensively used as flavoring and scenting agents in cosmetics, detergents, food and pharmaceutical products. They also display multiple biological activities in humans, such as anti-inflammatory, anti-microbial, antifungal and antiviral. Cannabis terpenes profiles define the aroma of each plant and share the same precursor (geranyl pyrophosphate) and the same synthesis location (glandular trichomes) as phytocannabinoids. The terpenoids most commonly found in Cannabis extracts include: limonine, myrcene, alpha-pinene, linalool, beta-caryophyllene, caryophyllene oxide, nerolidol, and phytol. Terpenes are mainly synthesized in two metabolic pathways: mevalonic acid pathway (a.k.a. HMG-CoA reductase pathway, which takes place in the cytosol) and MEP/DOXP pathway (a.k.a. The 2-C-methyl-D-erythritol 4-phosphate/l-deoxy-D-xylulose 5-phosphate pathway, non-mevalonate pathway, or mevalonic acid-independent pathway, which takes place in plastids). Geranyl pyrophosphate (GPP), which is used by cannabis plants to produce cannabinoids, is formed by condensation of dimethylallyl pyrophosphate (DMAPP) and isopentenyl pyrophosphate (IPP) via the catalysis of GPP synthase. Alternatively, DMAPP and IPP are ligated by FPP synthase to produce farnesyl pyrophosphate (FPP), which can be used to produce sesquiterpenoids. Geranyl pyrophospliate (GPP) can also be converted into monoterpenoids by limonene synthase.

Notably, a survey of the terpene profiles of several cannabis varieties has found that these terpenes express at high enough levels so as to have their own pharmacological effects and also to act in synergy with cannabinoids. The biochemical and phenomenological differences between different varieties of Cannabis, which are attributed to their unique relative cannabinoid and terpene ratios is known as the “entourage effect,” and is generally considered to result in plants providing advantages over only using the natural products that are isolated from them. These advantages include synergy with THC, the primary active ingredient, and also mitigation of side effects from THC. However, administration of cannabinoids and terpene compositions is limited by traditional routes of administration, such as smoking, vaping, or food-based infusions—which are not suitable for most traditional consumers. In some cases a consumer may wish to include a topical administration for ease of use, control of dose, and continuous delivery of therapeutic and cosmetic formulations of cannabinoids and/or terpene.

### SUMMARY OF THE INVENTION

The present invention includes systems. methods, and compositions for providing phytochemical infused cosmetics. In a preferred embodiment, the phytochemical infused cosmetics of the invention include color cosmetics containing one or more active ingredients selected from: a cannabinoid, a terpene, an endocannabinoid, or a combination of the same. In other preferred embodiments, the phytochemical infused cosmetics can be applied to the dermis of a user and provide a therapeutic, or recreational dose of one or more phytochemicals, namely a cannabinoid, a terpene, an endocannabinoid, or a combination of the same. In still other embodiments, the therapeutic dose can be for the treatment of a disease or condition for which the phytochemical can have a beneficial effect. In one preferred embodiment, the therapeutic dose can be a micro-dose, and/or a time-release dose configured to be administered over a pre-determined length of time.

Additional aspects of the invention may become evident based on the specification and claims presented below.

### DETAILED DESCRIPTION OF THE INVENTION

In one embodiment the present invention includes a phytochemical infused color cosmetic composition. In a preferred embodiment, the infused color cosmetic composition of the invention can include a quantity of water combined with a secondary component selected from: an emulsifier, a preservative, a thickener, an emollient, a color additive, a fragrance, and a pH stabilizer to form a color cosmetic composition, which further includes one or more active compounds selected from: a cannabinoid; and endocannabinoid, a terpene, or a combination of the same. In this preferred embodiment, the infused color cosmetic can include, but not be limited to: lipstick, lip oil, lip gloss, lip balm, foundation, mascara, brow gel, eyeliner, face primer, eye shadow, concealer, blush, lip liner, bronzer, eye primer, and face powder.

As used herein, the terms “cosmetic product(s)” and “cosmetic composition(s)” (also referred to as a composition of the invention) are those used to treat or care for, or somehow moisturize, improve, or clean the skin. The “cosmetic product(s)” and “cosmetic composition(s)” include, but are not limited to, moisturizers, personal cleansing products, makeup bases, foundations, occlusive drug delivery patches, nail polish, powders, wipes, hair conditioners, skin treatment emulsions, shaving creams and the like, and specifically include both functional and decorative cosmetic products.

As used herein, the term “color cosmetic(s),” and “colored cosmetic composition(s)” on the other hand, specifically refer to products that are applied to the face for the purpose of enhancing or changing its color. These are typically used to create a desired makeup look and include items such as foundation, concealer, blush, eyeshadow, lipstick, and other makeup products. Color cosmetics are primarily focused on adding color, texture, and definition to the features of the face, emphasizing certain areas or creating specific effects.

In a preferred embodiment, the cosmetic composition of the invention includes an emulsifier. As used herein, an emulsifier can include, but not be limited to: C₁₂-C₂₂ fatty acid esters with glycols, glycerin, pentaerythritol, sorbitol and saccharose, polyoxyethylene glycol derivativs, polyglyceryl derivative, alkyl sulphates, orto-phosphoric acid esters, amine salts, salts of quaternary ammonium, pyridine salts, betaine, lecithin, casein, gelatin, whey protein, starch, cellulose, rubber, slimes, and pectins, lecithins sterol, waxes, lanolin, bentonite, silica gel, magnesium carbonate, methylcellulose, carboxymethylcellulose, sulphated oils and fats, silicone copolymers, ionic and non-ionic surfactants, polyglycerol derivatives: polyglyceryl-3-polyricinoleate, polyglyceryl-4-diisostearate, or polyglyceryl-3-pentaolivate, ethoxylated fatty alcohols, Ethoxylated fatty acid esters, ethoxylated sorbitan fatty esters, ethoxylated glycerin monoesters, ethoxylated castor oil derivatives, ethoxylated derivatives of rapeseed oil, ethoxylated lanolin, ethoxylated polyglyceryl esters, alkyl polyglycosides, isethionates, alkyl phosphate salts, of a combination of any of the aforementioned.

In a preferred embodiment, the cosmetic composition of the invention includes a preservative. As used herein, a preservative can include, but not be limited to: Triclosan, DMDM hydantoin, Formaldehyde, Methylisothiazolinone, Methylchloroisothiazolinone, Phenoxyethanol, Methylparaben, Ethylparaben, Propylparaben, Butylparaben, Isobutylparaben, Benzoic Acid, Sorbic Acid, Salicylic Acid, sorbic acid (2,4-hexadienoic) s, potassium sorbate, benzoic acid, sodium benzoate, p-hydroxy benzoic acid, parabens, methyl Pparaben, alcohols, phenols, 2-phenoxyethanol, Bronopol, Germall, quaternary ammonium salts, methenamine 3-chloroallylochloride, dehydroacetic acid, anisic acid, phenylethyl alcohol, essential oils containing phenols, such as thymol, eugenol or carvacrol, thyme oil (Thymus vulgaris), clove oil (Syzygium aromaticum), tea tree oil (Melaleuca alternifolia), passiflora incarnata oil (Passiflora incarnata), lavender oil (Lavandulla officinallis), cinamon oil (Cinnamomum ceylanicum), sage oil (Salvia sclarea), witch-hazel oil (Hamamelis Virginiana L.), di- and triterpenoids, tannins, or polyphenols, extracts from thyme (Thymus Vulgaris), goldenseal (Hydrastis Canadensis Citrus), citron (Medica Limonum), lavender (Lavandula Augustifolia), Zeylanicum Bark), rosemary (Rosmarinus Officinalis), olive (Olea Europaea), oregano (Origanum Vulgare), mint (Mentha Piperita), or a combination of the same.

In a preferred embodiment, the cosmetic composition of the invention includes a thickener. As used herein, a thickener can include, but not be limited to: a lipid, Cetyl alcohol, Stearic acid and Carnauba wax, Hydroxyethyl cellulose, Guar gum, Xanthan gum, Gelatin, Magnesium aluminium silicate, Silica and Bentonite, Carbomer, Cetyl palmitateand, and Ammonium acryloyldimethyltaurate, or a combination of the same.

In a preferred embodiment, the cosmetic composition of the invention includes an emollient. As used herein, an emollient can include, but not be limited to: paraffin oil, petrolatum, squalene, dicapryl ether, and isohexadecane, Isopropyl myristate, isopropyl isostearate, silicone oil, and caprylic/capric triglycerides, Wheat germ oil, shea butter, octyldecanol, PPG-15 Stearyl Ether, and C₁₂-C₁₅ Alkyl Benzoatepolyglycerol derivatives, polyglyceryl-3-polyricinoleate, polyglyceryl-4-diisostearate, or polyglyceryl-3-pentaolivate, fatty acid monoesters, sorbitan monoolivate, glyceryl oleate, and glyceryl stearate, or a combination of the same.

In a preferred embodiment, the cosmetic composition of the invention includes an color additive. A color additive is a dye, pigment, or other substance, whether derived from a plant, animal, mineral, or other source, which imparts a color when added to a food, drug, cosmetic, or the human body. Color additive are regulated by the U.S. Food and Drug Administration and are listed at the following, being incorporated herein by reference: fda.gov/cosmetics/cosmetic-ingredient-names/color-additives-permitted-use-cosmetics.

In a preferred embodiment, the cosmetic composition of the invention includes a fragrance. In certain embodiments, the fragrance ca include a terpene, while in certain embodiments the terpene of the composition and a fragrance are the same or different compounds. In still further embodiments, the terpene of the composition is preferably a terpene from Cannabis. As used herein, a fragrance can include, but not be limited to: Linalool, Citronellol, Cinnamal, Limonene, Geranoil, Eugenoil, Essential oils, or a combination of the same.

In a preferred embodiment, the cosmetic composition of the invention includes a pH stabilizer, also referred to as a pH adjuster. As used herein, a pH stabilizer can include, but not be limited to: Sodium phytate, Propanediol, BHT, EDTA, Soy lecithin, Glucose oxidase, citric acid, tartaric acid, lactic acid, malic acid, succinic acid, fumaric acid, maleic acid, pyrophosphoric acid, gluconic acid, glucuronic acid, benzoic acid, AMPD, amino acids, sodium hydroxide, triethanolamine (TEA), or a combination of the same.

The invention may further include transdermal composition having one or more permeation enhancers to facilitate transfer of the phytochemical, and preferably a cannabinoid, terpene, or endocannabinoid, across a dermal layer. In a preferred embodiment, a permeation enhancer may include one or more of the following: propylene glycol monolaurate, diethylene glycol monoethyl ether, an oleoyl macrogolglyceride, a caprylocaproyl macrogolglyceride, an oleyl alcohol, or a combination of the same.

As noted above, the phytochemical infused color cosmetic compositions of the present invention are useful for a variety of therapeutic applications. For example, the compounds are useful for treating or alleviating symptoms of diseases and disorders involving CB1 and CB2 receptors, including appetite loss, nausea and vomiting, pain, multiple sclerosis and epilepsy. For example, they may be used to treat pain (i.e. as analgesics) in a variety of applications including but not limited to pain management. In additional embodiments, such modified cannabinoids compounds may be used as an appetite suppressant. As such, in one embodiment the present invention includes a method of treating a disease or condition, comprising the step of administering a therapeutically effective amount of an infused cosmetic of containing a quantity of water combined with a secondary component selected from: an emulsifier, a preservative, a thickener, an emollient, a color additive, a fragrance, and a pH stabilizer to form a color cosmetic composition, which further includes one or more active compounds selected from: a cannabinoid; and endocannabinoid, a terpene, or a combination of the same. In a preferred embodiment, the infused cosmetic can be applied directly to the subject's skin, and may further include a permeability enhancer to aid the transport of the active ingredient across the subject's dermal layer.

Implementation may generally involve identifying subjects suffering from the indicated disorders and administering the cosmetic compositions of the present invention, preferably through dermal application. The exact dosage of the phytochemical active ingredient 1 to be administered may vary depending on the age, gender, weight and overall health status of the individual patient, as well as the precise etiology of the disease. However, in general, for administration in mammals (e.g. humans), dosages in the range of from about 0.01 to about 300 mg of compound per kg of body weight per 24 hr., and more preferably about 0.01 to about 100 mg of compound per kg of body weight per 24 hr., are effective.

It should be noted that for any of the above compositions, unless otherwise stated, an effective amount of active compound, also referred to as the phytochemical, may include amounts between: .01 mg to .1 mg; .01 mg to .5 mg; .01 mg to 1 mg; .01 mg to 5 mg; .01 mg to 10 mg; .01 mg to 25 mg; .01 mg to 50 mg; .01 mg to 75 mg; .01 mg to 100 mg; .01 mg to 125 mg; .01 mg to 150 mg; .01 mg to 175 mg; .01 mg to 200 mg; .01 mg to 225 mg; .01 mg to 250 mg; .01 mg to 275 mg; .01 mg to 300 mg; .01 mg to 225 mg; .01 mg to 350 mg; .01 mg to 375 mg; .01 mg to 400 mg; .01 mg to 425 mg; .01 mg to 450 mg; .01 mg to 475 mg; .01 mg to 500 mg; .01 mg to 525 mg; .01 mg to 550 mg; .01 mg to 575 mg; .01 mg to 600 mg; .01 mg to 625 mg; .01 mg to 650 mg; .01 mg to 675 mg; .01 mg to 700 mg; .01 mg to 725 mg; .01 mg to 750 mg; .01 mg to 775 mg; .01 mg to 800 mg; .01 mg to 825 mg; .01 mg to 950 mg; .01 mg to 875 mg; .01 mg to 900 mg; .01 mg to 925 mg; .01 mg to 950 mg; .01 mg to 975 mg; .01 mg to 1000 mg; .01 mg to 2000 mg; .01 mg to 3000 mg; .01 mg to 4000 mg; 01 mg to 5000 mg; .01 mg to .1 mg/kg.; .01 mg to .5 mg/kg; 01 mg to 1 mg/kg; .01 mg to 5 mg/kg; .01 mg to 10 mg/kg; .01 mg to 25 mg/kg; .01 mg to 50 mg/kg; .01 mg to 75 mg/kg; and .01 mg to 100 mg/kg.

In one embodiment, the phytochemical infused color cosmetics of the invention can be used to treat a disease or condition selected from: obesity, post-traumatic stress syndrome, anorexia, nausea, emesis, pain, wasting syndrome, HIV-wasting, chemotherapy induced nausea and vomiting, alcohol use disorders, anti-tumor, amyotrophic lateral sclerosis, glioblastoma multiforme, glioma, increased intraocular pressure, glaucoma, cannabis use disorders, Tourette's syndrome, dystonia, multiple sclerosis, inflammatory bowel disorders, arthritis, dermatitis, Rheumatoid arthritis, systemic lupus erythematosus, anti-inflammatory, anti-convulsant, anti-psychotic, anti-oxidant, neuroprotective, anti-cancer, immunomodulatory effects, peripheral neuropathic pain, neuropathic pain associated with post-herpetic neuralgia, diabetic neuropathy, shingles, burns, actinic keratosis, oral cavity sores and ulcers, post-episiotomy pain, psoriasis, pruritis, contact dermatitis, eczema, bullous dermatitis herpetiformis, exfoliative dermatitis, mycosis fungoides, pemphigus, severe erythema multiforme (e.g., Stevens-Johnson syndrome), seborrheic dermatitis, ankylosing spondylitis, psoriatic arthritis, Reiter's syndrome, gout, chondrocalcinosis, joint pain secondary to dysmenorrhea, fibromyalgia, musculoskeletal pain, neuropathic-postoperative complications, polymyositis, acute nonspecific tenosynovitis, bursitis, epicondylitis, post-traumatic osteoarthritis, synovitis, and juvenile rheumatoid arthritis.

By “treating” the present inventors mean that the compound is administered in order to alleviate symptoms of the disease or disorder being treated. Those of skill in the art will recognize that the symptoms of the disease or disorder that is treated may be completely eliminated or may simply be lessened. Further, the compounds may be administered in combination with other drugs or treatment modalities.

As used herein, a “cannabinoid” is a chemical compound (such as cannabinol, THC or cannabidiol) that is found in the plant species Cannabis among others like: Echinacea; Acmella Oleracea; Helichrysum umbraculigerum; Radula marginata (Liverwort) and Theobroma cacao, and metabolites and synthetic analogues thereof that may or may not have psychoactive properties. Cannabinoids therefore include (without limitation) compounds (such as THC) that have high affinity for the cannabinoid receptor (for example Ki<250 nM), and compounds that do not have significant affinity for the cannabinoid receptor (such as cannabidiol, CBD). Cannabinoids also include compounds that have a characteristic dibenzopyran ring structure (of the type seen in THC) and cannabinoids which do not possess a pyran ring (such as cannabidiol). Hence a partial list of cannabinoids includes THC, CBD, dimethyl heptylpentyl cannabidiol (DMHP-CBD), 6,12-dihydro-6-hydroxy-cannabidiol (described in U.S. Pat. No. 5,227,537, incorporated by reference); (3S,4R)-7-hydroxy-Δ6-tetrahydrocannabinol homologs and derivatives described in U.S. Pat. No. 4,876,276, incorporated by reference; (+)-4-[4-DMH-2,6-diacetoxy-phenyl]-2-carboxy-6,6-dimethylbicyclo[3.1.1]hept-2-en, and other 4-phenylpinene derivatives disclosed in U.S. Pat. No. 5,434,295, which is incorporated by reference; and cannabidiol (−)(CBD) analogs such as (−)CBD-monomethylether, (−)CBD dimethyl ether; (−)CBD diacetate; (−)3′-acetyl-CBD monoacetate; and ±AF11, all of which are disclosed in Consroe et al., J. Clin. Phannacol. 21:428S-436S, 1981, which is also incorporated by reference. Many other cannabinoids are similarly disclosed in Agurell et al., Pharmacol. Rev. 38:31-43, 1986, which is also incorporated by reference.

Examples of cannabinoids are tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol,trihydroxy-delta-9-tetrahydrocannabinol, and cannabinol. Examples of cannabinoids within the context of this disclosure include tetrahydrocannabinol and cannabidiol. The term “cannabinoid” may also include different modified forms of a cannabinoid such as a hydroxylated cannabinoid or cannabinoid carboxylic acid. For example, if a UGT were to be capable of glycosylating a cannabinoid, it would include the term cannabinoid as defined elsewhere, as well as the aforementioned modified forms. It may further include multiple glycosylation moieties.

The term “endocannabinoid” refers to compounds including arachidonoyl ethanolamide (anandamide, AEA), 2-arachidonoyl ethanolamide (2-AG), 1-arachidonoyl ethanolamide (1-AG), and docosahexaenoyl ethanolamide (DHEA, synaptamide), oleoyl ethanolamide (OEA), eicsapentaenoyl ethanolamide, prostaglandin ethanolamide, docosahexaenoyl ethanolamide, linolenoyl ethanolamide, 5(Z),8(Z),1 1 (Z)-eicosatrienoic acid ethanolamide (mead acid ethanolamide), heptadecanoul ethanolamide, stearoyl ethanolamide, docosaenoyl ethanolamide, nervonoyl ethanolamide, tricosanoyl ethanolamide, lignoceroyl ethanolamide, myristoyl ethanolamide, pentadecanoyl ethanolamide, palmitoleoyl ethanolamide, docosahexaenoic acid (DHA). Particularly preferred endocannabinoids are AEA, 2-AG, 1-AG, and DHEA.

Terpenes, which may be used interchangeably with the term terpenoids, are a large and diverse class of naturally occurring organic chemicals. Terpenes are essential for plant metabolism, influencing general development, herbivory defense, pollination and stress response. These compounds have been extensively used as flavoring and scenting agents in cosmetics, detergents, food and pharmaceutical products. They also display multiple biological activities in humans, such as anti-inflammatory, anti-microbial, antifungal and antiviral.

The term “terpene” refers to the large and diverse class of organic compounds produced by a variety of plants, including ('annabis plants. (As used herein, the terms “terpene” and “terpenoid” are used interchangeably.) When terpenes are modified chemically, such as by oxidation or rearrangement of the carbon skeleton, the resulting compounds are generally referred to as “terpenoids.” The structure of terpenes are built with isoprenes, which are 5 carbon structures. Flavonoids are generally considered to be 15 carbon structures with two phenyl rings and a heterocyclic ring. So, there could be an overlap in which a flavonoid could be considered a terpene. However, not all terpenes could be considered flavonoids. Cannabis terpenes profiles define the aroma of each plant and share the same precursor (geranyl pyrophosphate) and the same synthesis location (glandular trichomes) as phytocannabinoids. The terpenoids most commonly found in Cannabis extracts include: limonine, myrcene, alpha-pinene, linalool, beta-caryophyllene, caryophyllene oxide, nerolidol, and phytol.

Within the context of the inventive technology, the term terpene includes: Flemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives. Derivatives of terpenes include Terpenoids in their forms of hemiterpenoids, monoterpenoids, sesquiterpenoids, sesterterpenoid, sesquarterpenoids, tetraterpenoids, Triterpenoids, tetraterpenoids, Polyterpenoids, isoprenoids, and steroids. They may be forms: α-, β-, γ-, oχo0-, isomers, or combinations thereof.

Examples of terpenes within the context of the inventive technology include: 7,8-dihydroionone, Acetanisole, Acetic Acid, Acetyl Cedrene, Anethole, Anisole, Benzaldehyde, Bergamotene (α-cis-Bergamotene) (a-trans-Bergamotene), Bisabolol (β-Bisabolol), Borneol, Bornyl Acetate, Butanoic/Butyric Acid, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (a-Cedrene Epoxide), Cedrol, Cembrene, Chlorogenic Acid, Cinnamaldehyde (α-amyl-Cinnamaldehyde) (a-hexyl-Cinnamaldehyde), Cinnamic Acid, Cinnamyl Alcohol, Citronellal, Citronellol, Cryptone, Curcumene (α-Curcumene) (γ-Curcumene), Decanal, Dehydrovomifoliol, Diallyl Disulfide, Dihydroactinidiolide, Dimethyl Disulfide, Eicosane/lcosane, Elemene (β-Elemene), Estragole, Ethyl acetate, Ethyl Cinnamate, Ethyl maltol, Eucalyptol/1,8-Cineole, Eudesmol (a-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1 (10),11-diene, Guaiacol, Guaiene (a-Guaiene), Gurjunene (α-Gurjunene), Herniarin, Flexanaldehyde, Flexanoic Acid, Humulene (a-Humulene) (β-Humulene), lonol (3-oxo-a-ionol) (β-IoηoI), lonone (a-lonone) (β-lonone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, a-Sinensal, β-Sinensal, β-Sitosterol, Squalene, Taxadiene, Terpin hydrate, Terpineol, Terpine-4-ol, α-Terpinene, y-Terpinene, Terpinolene, Thiophenol, Thujone, Thymol, a-Tocopherol, Tonka Undecanone, Undecanal, Valeraldehyde/Pentanal, Verdoxan, a-Ylangene, Umbelliferone, or Vanillin. Diterpenes: oridonin, phytol, and isophytol. Triterpenes: ursolic acid, oleanolic acid, among those listed elsewhere.

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein. The terms “approximately” and “about” refer to a quantity, level, value, or amount that varies by as much as 30%, or in another embodiment by as much as 20%, and in a third embodiment by as much as 10% to a reference quantity, level, value or amount.

As used herein, the term “comprise,” or variations thereof such as “comprises” or “comprising,” are to be read to indicate the inclusion of any recited integer (e.g., a feature, element, characteristic, property, method/process step or limitation) or group of integers (e.g., features, element, characteristics, properties, method/process steps or limitations) but not the exclusion of any other integer or group of integers. Thus, as used herein the term “comprising” is inclusive or open-ended and does not exclude additional, unrecited integers or method/process steps.

1. An infused color cosmetic composition comprising:
a quantity of water combined with a secondary component selected from: an emulsifier, a preservative, a thickener, an emollient, a color additive, a fragrance, and a pH stabilizer to form a color cosmetic composition; and
one or more active compounds selected from: a cannabinoid; an endocannabinoid, a terpene, or a combination of the same.

2. The infused cosmetic of claim 1, wherein said cannabinoid is selected from: tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, trihydroxy-delta-9-tetrahydrocannabinol, and cannabinol.

3. The infused cosmetic of claim 1, wherein said terpene is selected from: 7,8-dihydroionone, Acetanisole, Acetic Acid, Acetyl Cedrene, Anethole, Anisole, Benzaldehyde, Bergamotene (α-cis-Bergamotene) (a-trans-Bergamotene), Bisabolol (β-Bisabolol), Borneol, Bornyl Acetate, Butanoic/Butyric Acid, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (a-Cedrene Epoxide), Cedrol, Cembrene, Chlorogenic Acid, Cinnamaldehyde (α-amyl-Cinnamaldehyde) (a-hexyl-Cinnamaldehyde), Cinnamic Acid, Cinnamyl Alcohol, Citronellal, Citronellol, Cryptone, Curcumene (α-Curcumene) (γ-Curcumene), Decanal, Dehydrovomifoliol, Diallyl Disulfide, Dihydroactinidiolide, Dimethyl Disulfide, Eicosane/lcosane, Elemene (β-Elemene), Estragole, Ethyl acetate, Ethyl Cinnamate, Ethyl maltol, Eucalyptol/1,8-Cineole, Eudesmol (a-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1 (10),11-diene, Guaiacol, Guaiene (a-Guaiene), Gurjunene (α-Gurjunene), Herniarin, Flexanaldehyde, Flexanoic Acid, Humulene (a-Humulene) (β-Humulene), lonol (3-oxo-a-ionol) (β-IonoI), lonone (a-lonone) (β-lonone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, α-Longipinene, Lycopene, Menthol, Methyl butyrate, 3-Mercapto-2-Methylpentanal, Mercaptan/Thiols, β-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, a-Sinensal, β-Sinensal, β-Sitosterol, Squalene, Taxadiene, Terpin hydrate, Terpineol, Terpine-4-ol, α-Terpinene, y-Terpinene, Terpinolene, Thiophenol, Thujone, Thymol, a-Tocopherol, Tonka Undecanone, Undecanal, Valeraldehyde/Pentanal, Verdoxan, a-Ylangene, Umbelliferone, or Vanillin, oridonin, phytol, and isophytol, ursolic acid, oleanolic acid

4. The infused cosmetic of claim 1, wherein said endocannabinoid is selected from: arachidonoyl ethanolamide (anandamide, AEA), 2-arachidonoyl ethanolamide (2-AG), 1-arachidonoyl ethanolamide (1-AG), and docosahexaenoyl ethanolamide (DHEA, synaptamide), oleoyl ethanolamide (OEA), eicsapentaenoyl ethanolamide, prostaglandin ethanolamide, docosahexaenoyl ethanolamide, linolenoyl ethanolamide, 5(Z),8(Z), 1 1 (Z)-eicosatrienoic acid ethanolamide (mead acid ethanolamide), heptadecanoul ethanolamide, stearoyl ethanolamide, docosaenoyl ethanolamide, nervonoyl ethanolamide, tricosanoyl ethanolamide, lignoceroyl ethanolamide, myristoyl ethanolamide, pentadecanoyl ethanolamide, palmitoleoyl ethanolamide, docosahexaenoic acid (DHA).

5. The infused cosmetic of claim 1, further comprising a permeation enhancer.

6. The infused cosmetic of claim 5, wherein said permeation enhancer is selected from: propylene glycol monolaurate, diethylene glycol monoethyl ether, an oleoyl macrogolglyceride, a caprylocaproyl macrogolglyceride, and oleyl alcohol.

7. The infused cosmetic of claim 1, wherein said color cosmetic is selected from: lipstick, lip oil, lip gloss, lip balm, foundation, mascara, brow gel, eyeliner, face primer, eye shadow, concealer, blush, lip liner, bronzer, eye primer, and face powder.

8-12. (canceled)

13. A method of producing an infused color cosmetic composition comprising the step of:
establishing a quantity of water combined with a secondary component selected from: an emulsifier, a preservative, a thickener, an emollient, a color additive, a fragrance, and a pH stabilizer to form a color cosmetic composition; and
adding to the quantity of water, one or more active compounds selected from: a cannabinoid; an endocannabinoid, a terpene, or a combination of the same.

14. The method of claim 13, wherein said cannabinoid is selected from: tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid, delta-9-tetrahydrocannabivarin, delta-9-tetrahydrocannabiorcolic acid, delta-9-tetrahydrocannabiorcol, delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, delta-9-cis-tetrahydrocannabinol, 3, 4, 5, 6-tetrahydro-7-hydroxy-alpha-alpha-2-trimethyl-9-n-propyl-2, 6-methano-2H-1-benzoxocin-5-methanol-cannabiripsol, trihydroxy-delta-9-tetrahydrocannabinol, and cannabinol.

15. The method of claim 13, wherein said terpene is selected from: 7,8-dihydroionone, Acetanisole, Acetic Acid, Acetyl Cedrene, Anethole, Anisole, Benzaldehyde, Bergamotene (α-cis-Bergamotene) (a-trans-Bergamotene), Bisabolol (β-Bisabolol), Borneol, Bornyl Acetate, Butanoic/Butyric Acid, Cadinene (α-Cadinene) (γ-Cadinene), Cafestol, Caffeic acid, Camphene, Camphor, Capsaicin, Carene (Δ-3-Carene), Carotene, Carvacrol, Carvone, Dextro-Carvone, Laevo-Carvone, Caryophyllene (β-Caryophyllene), Caryophyllene oxide, Castoreum Absolute, Cedrene (α-Cedrene) (β-Cedrene), Cedrene Epoxide (a-Cedrene Epoxide), Cedrol, Cembrene, Chlorogenic Acid, Cinnamaldehyde (α-amyl-Cinnamaldehyde) (a-hexyl-Cinnamaldehyde), Cinnamic Acid, Cinnamyl Alcohol, Citronellal, Citronellol, Cryptone, Curcumene (α-Curcumene) (γ-Curcumene), Decanal, Dehydrovomifoliol, Diallyl Disulfide, Dihydroactinidiolide, Dimethyl Disulfide, Eicosane/lcosane, Elemene (β-Elemene), Estragole, Ethyl acetate, Ethyl Cinnamate, Ethyl maltol, Eucalyptol/1,8-Cineole, Eudesmol (a-Eudesmol) (β-Eudesmol) (γ-Eudesmol), Eugenol, Euphol, Farnesene, Farnesol, Fenchol (β-Fenchol), Fenchone, Geraniol, Geranyl acetate, Germacrenes, Germacrene B, Guaia-1 (10),11-diene, Guaiacol, Guaiene (a-Guaiene), Gurjunene (α-Gurjunene), Herniarin, Flexanaldehyde, Flexanoic Acid, Humulene (a-Humulene) (α-Humulene), lonol (3-oxo-a-ionol) (β-IoηoI), lonone (a-lonone) (B-lonone), Ipsdienol, Isoamyl acetate, Isoamyl Alcohol, Isoamyl Formate, Isoborneol, Isomyrcenol, Isopulegol, Isovaleric Acid, Isoprene, Kahweol, Lavandulol, Limonene, γ-Linolenic Acid, Linalool, Longifolene, a-Longipinene, Lycopene, Menthol, Methyl butyrate, 3-Mercapto-2-Methylpentanal, Mercaptan/Thiols, B-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (β-Myrcene), γ-Muurolene, Nepetalactone, Nerol, Nerolidol, Neryl acetate, Nonanaldehyde, Nonanoic Acid, Ocimene, Octanal, Octanoic Acid, P-cymene, Pentyl butyrate, Phellandrene, Phenylacetaldehyde, Phenylethanethiol, Phenylacetic Acid, Phytol, Pinene, β-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, α-Selinene, a-Sinensal, β-Sinensal, β-Sitosterol, Squalene, Taxadiene, Terpin hydrate, Terpineol, Terpine-4-ol, α-Terpinene, γ-Terpinene, Terpinolene, Thiophenol, Thujone, Thymol, a-Tocopherol, Tonka Undecanone, Undecanal, Valeraldehyde/Pentanal, Verdoxan, a-Ylangene, Umbelliferone, or Vanillin, oridonin, phytol, and isophytol, ursolic acid, oleanolic acid

16. The method of claim 13, wherein said endocannabinoid is selected from: arachidonoyl ethanolamide (anandamide, AEA), 2-arachidonoyl ethanolamide (2-AG), 1-arachidonoyl ethanolamide (1-AG), and docosahexaenoyl ethanolamide (DHEA, synaptamide), oleoyl ethanolamide (OEA), eicsapentaenoyl ethanolamide, prostaglandin ethanolamide, docosahexaenoyl ethanolamide, linolenoyl ethanolamide, 5(Z),8(Z), 1 1 (Z)-eicosatrienoic acid ethanolamide (mead acid ethanolamide), heptadecanoul ethanolamide, stearoyl ethanolamide, docosaenoyl ethanolamide, nervonoyl ethanolamide, tricosanoyl ethanolamide, lignoceroyl ethanolamide, myristoyl ethanolamide, pentadecanoyl ethanolamide, palmitoleoyl ethanolamide, docosahexaenoic acid (DHA).

17. The method of claim 13, further comprising a permeation enhancer.

18. The method of claim 17, wherein said permeation enhancer is selected from: propylene glycol monolaurate, diethylene glycol monoethyl ether, an oleoyl macrogolglyceride, a caprylocaproyl macrogolglyceride, and oleyl alcohol.

19. The method of claim 13, wherein said color cosmetic is selected from: lipstick, lip oil, lip gloss, lip balm, foundation, mascara, brow gel, eyeliner, face primer, eye shadow, concealer, blush, lip liner, bronzer, eye primer, and face powder.

20. (canceled)
